(19) United States
(12) Patent Application Publication
(10) Pub. No.: US 2018/0008823 A1
(43) Pub. Date: Jan. 11, 2018

(54) SYSTEM AND METHOD FOR STIMULATING HAIR GROWTH

(71) Applicant: The General Hospital Corporation, Boston, MA (US)

(72) Inventors: Saiqa Khan, Boston, MA (US); Alexander Golberg, Boston, MA (US); Martin Yarmush, Newton, MA (US); William G. Austen, Jr., Weston, MA (US)

(21) Appl. No.: 15/546,357

(22) PCT Filed: Jan. 25, 2016
(86) PCT No.: PCT/US16/14673

§ 371 Date: Jul. 26, 2017

Related U.S. Application Data
(60) Provisional application No. 62/108,830, filed on Jan. 28, 2015.

(51) Int. Cl. A61N 1/32; A61K 8/98; A61N 1/04

(52) U.S. Cl. CPC A61N 1/326; A61K 8/985; A61N 1/0404

(57) ABSTRACT

A system and method are provided for stimulating hair growth of a user. The system includes a stimulating unit in communication with a control unit. The stimulating unit includes at least one electrode to be placed on a region of the user, a pulser configured to send electronic pulses to the electrode, and a power supply which supplies power to the pulser. Operation of the system includes the steps of placing the at least one electrode on a region of the user, relaying a plurality of pulses to the electrode, terminating the pulses when a predetermined number of pulses have been delivered, and removing the electrode from the region of the user.

### CROSS-REFERENCE TO RELATED APPLICATIONS

This application is based on, claims priority to, and incorporates herein by reference in its entirety, U.S. Provisional Patent Application Ser. No. 62/108,830, filed Jan. 28, 2015, and entitled “SYSTEM AND METHOD FOR STIMULATING HAIR GROWTH.”

### BACKGROUND OF THE INVENTION

Alopecia, or loss of hair, affects more than half of the population worldwide. Androgenic alopecia, the most common type, affects 50% of males over the age of 40, and 75% of females over the age of 65. According to 46% of women undergoing chemotherapy for breast cancer, alopecia was the most traumatic side effect.

The human hair cycle consists of three stages. During the first stage, anagen, new hair grows. During the transitional phase, catagen, cell division stops for two to three weeks. A third resting phase, telogen, lasts three to four months. During this phase, hairs are shed and the follicle remains dormant. Alopecia occurs when more follicles are in the telogen phase than the anagen phase.

Current therapeutic options include drugs and hair transplantation. Two drugs, minoxidil and finasteride, are currently approved for treatment of alopecia. Minoxidil is a topical drug which must be applied to the scalp twice a day in order to halt hair loss and stimulate new hair growth through vasodilation. Finasteride, a daily pill for androgenic alopecia, converts follicles into the anagen phase by inhibiting (Type II) 5-α reductase, an enzyme which converts testosterone to a hair loss stimulating androgen dihydrotestosterone (DHT). However, finasteride has not been proven to work on the hairline, or the frontal and temporal areas of the scalp. Furthermore, discontinuation of minoxidil or finasteride causes shedding of rescued hair and return to baldness.

An alternative treatment for alopecia is hair transplantation, one of the most common aesthetic procedures performed in the male population. Complete hair restoration is costly, may require multiple surgical procedures, can require eight months to one year for full results, and may cause significant donor site morbidity from scarring. Overall, current therapies are expensive, not consistently effective, lead to donor site morbidity, and require daily use to avoid recurrent alopecia.

### SUMMARY OF THE INVENTION

The present invention overcomes the above and other drawbacks by providing a system and method for stimulating hair growth using pulsed electric fields (PEF). The system and method provide for a non-invasive, easy-to-use, effective therapy that can be used at the home of a user.

In one aspect, the present invention provides a system for stimulating hair growth, the system including a stimulating unit and a control unit. The stimulating unit includes at least one electrode, a pulser in communication with the electrode that is configured to provide an electronic pulse to the electrode, and a power source configured to provide power to the pulser. The control unit is configured to communicate at least one stimulation parameter to the pulser, wherein the at least one stimulation parameter is selected to cause hair follicles proximate to the electrode to enter an anagen stage.

In an additional aspect, the present invention provides a method for stimulating hair growth, the method including the steps of placing at least one electrode of a stimulating unit on a region of a user, and delivering at least one electric pulse to the at least one electrode according to at least one pulse parameter, wherein the at least one pulse parameter is selected to cause hair follicles proximate to the region of the user to enter an anagen stage. The method further includes the steps of terminating the delivery of the electric pulses when a predetermined number of pulses have been delivered, wherein the predetermined number is selected to cause hair follicles proximate to the region of the user to enter the anagen stage, and removing the at least one electrode from the region of the user.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a block diagram of a stimulating system.

FIG. 2 is an example process flow chart for operating the stimulating system.

FIG. 3A is a histological sample of a dorsum of a rat before treatment.

FIG. 3B is a histological sample of the dorsum of the rate after treatment.

FIG. 3C is a pictorial representation of the dorsum of the rate before treatment.

FIG. 3D is a pictorial representation of the dorsum of the rat after treatment.

### DETAILED DESCRIPTION OF THE INVENTION

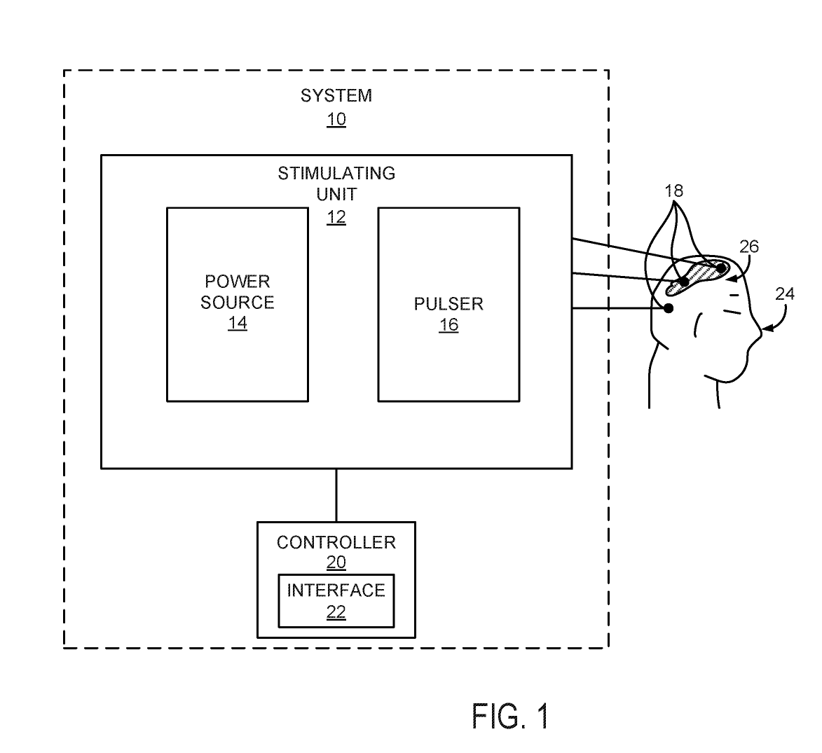

Referring to FIG. 1, a stimulating system 10 for stimulating hair follicles to the anagen phase is illustrated. As can be seen in the block diagram of FIG. 1, the stimulating system 10 includes a stimulating unit 12 power source 14, a pulser 16, and at least one electrode 18.

It is possible for the stimulating system 10 to include a control unit 20, for example a computer or commercially-available processor, in communication with the pulser 16. The control unit 20 may include a user interface 22 that allows for a user to input stimulation parameters for a pulse length, pulse frequency, number of pulses, voltage, and/or any other desirable stimulation parameters. In an alternative configuration, the control unit 20 may be incorporated within the stimulating unit 12. For example, if a set of stimulation parameters is found to be desirable, these parameters may be programmed into the control unit 20 such that no user input is needed. The stimulation parameters can be chosen such that hair follicles near the stimulating unit 12 enter the anagen state when the stimulating unit 12 is in use. Thus, as illustrated the electrodes 18 may be coupled to user 24, for example on the head or other area of the body where hair growth is desired. As will be described, the electrodes may be coupled directly to the user 24 or, optionally, conductive gels or additional materials 26 may be utilized. Such gels or other materials may assist with treating the skin/follicles, despite the presence of existing hair. Materials such as ultrasound gel or other substances may optionally include active ingredients, such as biotin and vitamins may be included. Such active ingredients may be used or coordinated with use of the above-described system 10, such that the operation of the facilitates delivery of active ingredients to the cells.

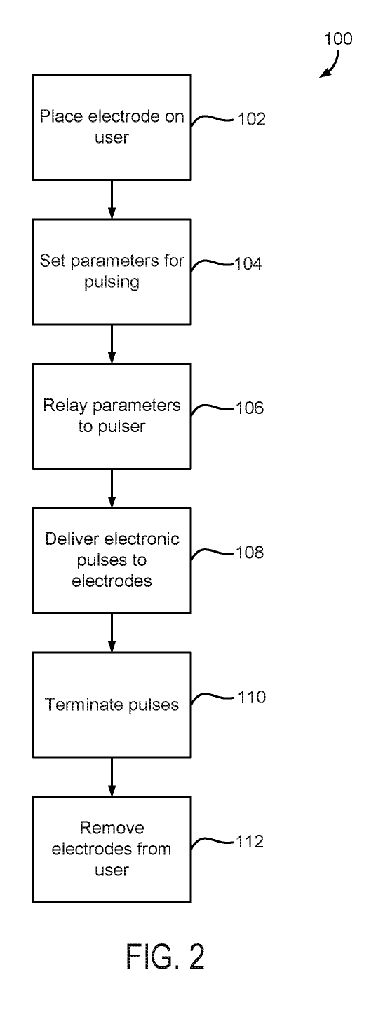

A non-limiting example for using the stimulating system 10 is shown in the process flow 100 of FIG. 2. The at least one electrode 18 is placed on a region of a user wherein hair growth in desired as shown in step 102, and stimulation parameters for pulsing are set as shown in step 104. The stimulation parameters are relayed to the pulser 16 as seen in step 106. The pulser 16 delivers electronic pulses to the at least one electrode 18 as seen in step 108. The electronic pulses end after, for example, a predetermined number of pulses as shown in step 110. The at least one electrode 18 can be removed from the user as seen in step 112. As will be described, the specific stimulation parameters and/or duration can be selected to cause hair follicles to enter the anagen stage and, thereby, cause or engage hair growth in hair follicles proximate to the at least one electrode 18.

To this end, the stimulating unit 12 to be incorporated into an article that is designed to locate the at least one electrode 18 proximate to a desired region for hair growth. For example the articles may be designed to cover the region of the user where hair growth is desired. As such, the stimulating unit 12 may be integral with a hat, helmet, or other acceptable articles when the user desires hair growth on the scalp or elsewhere on the body. To this end, the stimulating unit 12 may be integrated into a hand-held device, such as a wand or comb.

Further details, including specific and desired operational parameters will be set forth with respect to the following examples.

### EXAMPLE 1

In one example, fifteen Sprague Dawley rats were shaved and treated with PEF using two contact electrodes having a surface area of 1 cm². Three treatment sites and three control sites were randomized and tattooed onto the dorsum of each rat, for a total of six sites per rat. Two sites were assigned to the cranial aspect of the dorsum, two at the middle, and two at the caudal aspect of the dorsum to account for the wave-like pattern of rodent hair growth.

Six of the fifteen rats were tested using PEFs having 200 pulses, each having a voltage of 500V, a 70 us pulse length and a frequency of 2 Hz. The remaining nine rats were tested using a low-dose experiment following the Taguchi experimental design as shown in Table 1. A range of the stimulation parameters associated with the voltage, number of pulses, and pulse length were tested to determine the effect of each parameter on hair stimulation. Daily digital photography of the dorsa was obtained, and each rat was shaved 3-weeks after treatment. The rats were euthanized 1-month after treatment, and tissues were harvested for histological analysis.

TABLE 1
Taguchi Experimental Design

| Cage # | Rat # | Voltage (V) | Pulse Length (μs) | Pulse # |
|---|---|---|---|---|
| 1 | 1 | 30 | 10 | 100 |
| | 2 | 30 | 90 | 300 |
| | 3 | 30 | 270 | 900 |
| 2 | 4 | 90 | 10 | 300 |
| | 5 | 90 | 90 | 900 |
| | 6 | 90 | 270 | 100 |
| 3 | 7 | 270 | 10 | 900 |
| | 8 | 270 | 90 | 100 |
| | 9 | 270 | 270 | 300 |

The total number of hair follicles was counted, as well as the number of follicles in the anagen phase per histology section. The percentage of anagen follicles per site was then calculated. Data was analyzed using a t-test with p<0.05 considered statistically significant. Taguchi analysis was performed on the low dose data and ranks were assigned to stimulation parameters according to the results.

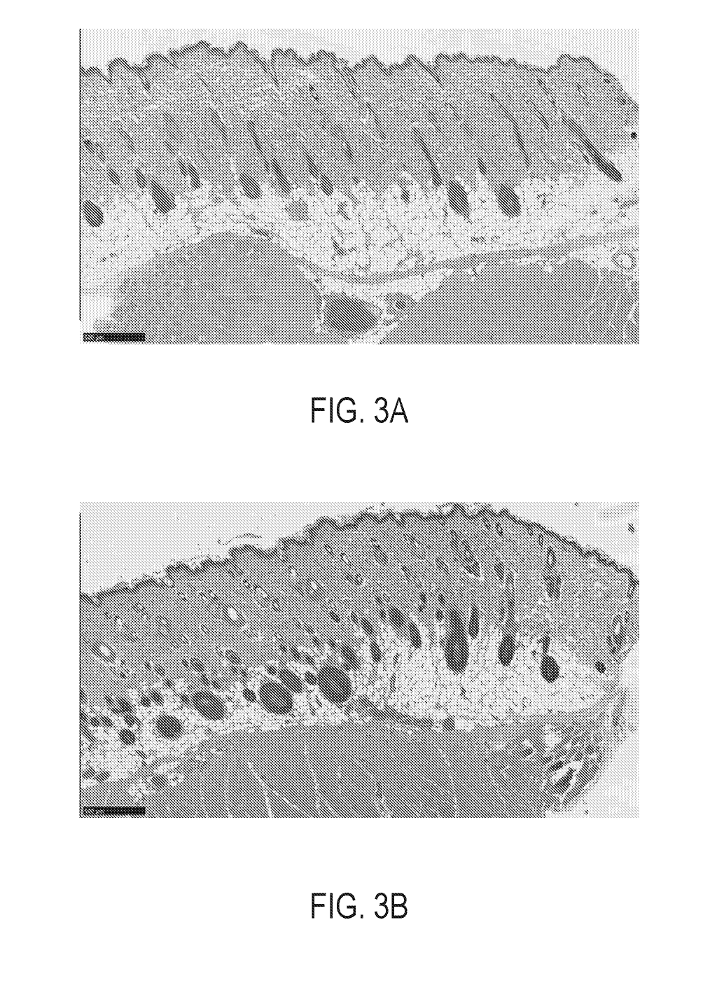

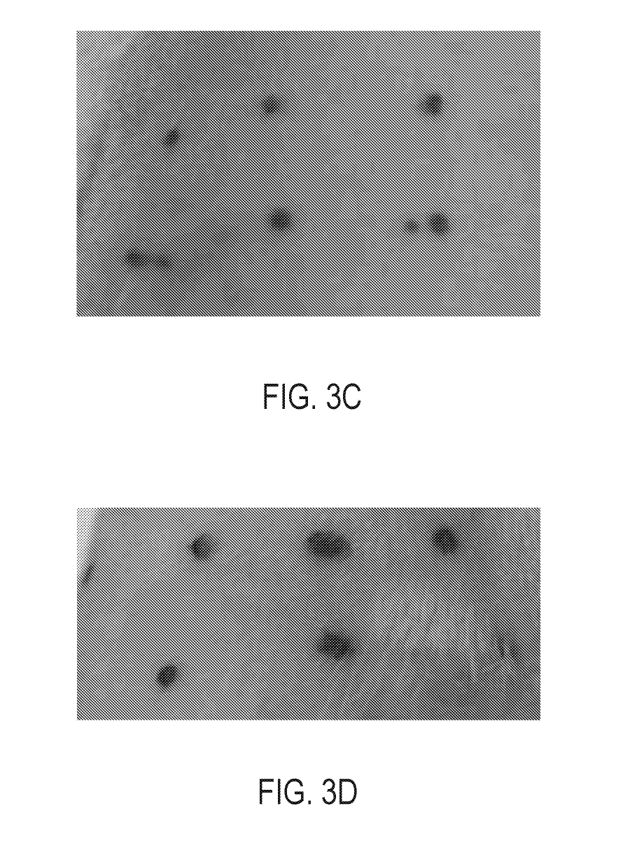

Referring to FIG. 3A showing a rat before treatment and 3B showing the rat after treatment, an increase in active anagen follicles after PEF treatment in the 500V, 200 pulse group of mice can be seen. Histological analysis correlated with the digital photography, shown in FIG. 3C showing the rat before treatment and FIG. 3D showing the rat after treatment. Treated sites demonstrated 15.8±9.78% of follicles in anagen compared to 4.6±9.13% of follicles in the anagen phase at control sites (p=0.0003).

Analysis of the hair growth related to the nine remaining rats in the Taguchi low-dose experiment demonstrated increased hair growth with treatment. A dose response was appreciated, and results indicated that increasing the voltage had the greatest effect on anagen stimulation. Table 2 below demonstrates the effect on average fold increase in percent anagen for each individual dose evaluated.

TABLE 2
Low-dose Experimental Results

| | | | Pulse | | Average Fold | |
|---|---|---|---|---|---|---|
| Cage | Rat | Voltage | Length | Pulse | Increase in | |
| # | # | (V) | (μs) | # | Percent Anagen | SEM |
| 1 | 1 | 30 | 10 | 100 | 4 | 3 |
| | 2 | 30 | 90 | 300 | 1 | 0 |
| | 3 | 30 | 270 | 900 | 6 | 6 |
| 2 | 4 | 90 | 10 | 300 | 5 | 3 |
| | 5 | 90 | 90 | 900 | 2 | 2 |
| | 6 | 90 | 270 | 100 | 4 | 3 |
| 3 | 7 | 270 | 10 | 900 | 6 | 3 |
| | 8 | 270 | 90 | 100 | 1 | 0 |
| | 9 | 270 | 270 | 300 | 9 | 4 |

As shown in Table 3, rats undergoing PEF treatment with stimulation parameters of 270 V, 300 pulses, and 270 μs pulse length duration induced an overall 5.05-fold increase in anagen follicles at treated sites as compared to controls. Treated sites demonstrated 45.55±18.07% of follicles in anagen, contrasting 9.02±6.00% of follicles in the anagen phase at control sites (p=0.0008). When adjusted per level to account for the fact that rodent hair grows in a wave-like fashion, a 9±4 fold increase in anagen was appreciated at treated sites. Digital photography correlated with histological findings, revealing defined square patches of hair at treated sites distinctly contrasting surrounding untreated skin, as can be seen in Table 3.

TABLE 3
270 V, 300 pulses, 270 μs

| | Treated Sites | Control Sites |
|---|---|---|
| Level | (% anagen) | (% anagen) |
| Cranial | 47.48 | 14.55 |
| Middle | 62.61 | 10.92 |
| Caudal | 26.55 | 1.583 |

As seen in Table 4, treatment with the stimulation parameters of 90 V, 300 pulses, and 10 μs pulse length duration resulted in a 4.03-fold increase in active anagen follicles at treated sites compared to controls. Treated sites revealed 33.15±6.75% anagen follicles whereas 8.23±7.66% of follicles were in the anagen phase at control sites (p=0.0134). When adjusted per level, a 5±3 fold increase in anagen was demonstrated at treated sites.

TABLE 4
90 V, 300 pulses, 10 μs

| | Treated Sites | Control Sites |
|---|---|---|
| Level | (% anagen) | (% anagen) |
| Cranial | 29.23 | 9.52 |
| Middle | 29.27 | 15.15 |
| Caudal | 40.94 | 3.72 |

Treatment with the stimulation parameters of 30 V, 300 pulses, and 90 μs pulse length duration demonstrated no significant statistical difference between percent anagen follicles at treated and control sites. A 1.26 fold increase in active anagen follicles at treated sites could be seen, with 17.54±28.11% at treated sites vs. 13.87±22.60% at control sites (p=0.8686).

However, with the stimulation parameters having a continued 30 V, together with an increased pulse number of 900 pulses and increased pulse length of 270 μs, a 6±6 average fold increase in percent anagen was seen. As can be seen in Table 5 below, a large amount of variability is seen in the percent anagen at the treated and control sites. Treated sites at levels 1 and 2 at this dose revealed lower percent anagen as compared to control sites. Only level 3 demonstrated an increase in percent anagen at the treated site as compared to the control site.

TABLE 5
30 V, 900 pulses, 270 μs

| | Treated Sites | Control Sites |
|---|---|---|
| Level | (% anagen) | (% anagen) |
| Cranial | 1.85 | 16.39 |
| Middle | 1.76 | 16.44 |
| Caudal | 63.49 | 3.81 |

The low-dose experiment demonstrated that a single treatment using PEF shifts resting follicles into active anagen resulting in a 3.4-fold increase in anagen follicles when treated with 500V, 200 pulses and 70 μs pulse length duration. This leads to dense patches of rapidly growing hair at treated sites. Voltage was found to be a particularly influential parameter for increasing hair growth. A single treatment at 270 V, 300 pulses, and 270 μs pulse length duration lead to a 5.05-fold increase in anagen follicles at treated sites. A single treatment at 90 V, 300 pulses, and 10 μs pulse length duration, resulted in a 4.03-fold increase in active anagen follicles at treated sites.

The goal of this experiment was to evaluate parameter optimization for lower doses. The Taguchi rank generated a parameter optimization such that voltage ranked highest, pulse length ranked second, and number of pulses ranked lowest regarding effect on hair stimulation.

Within the context of the above-described, non-limiting example, operational parameters of 270 V, 300 pulses, and 270 μs were formed as a potential “optimal” dose because effects below and above this dose are not as profound as the growth appreciated at this dose. Of course, variations in users and adjustments to other treatment parameters can lead to other operational parameter values being preferable for those circumstances. For example, the voltage parameter range, even within the context of this non-limiting example, may span from 30 volts to 500 volts or a greater range. Likewise, the pulses may range from 200 pulses to 900 pulses or a greater range.

Furthermore, the operational parameters may be desirably varied in the case of multiple treatments. For example, it is possible that the above-described therapy can be used once per month by the user to maintain or increase the effects. On the other hand, for some users one treatment is sufficient and regular touch-ups or maintenance is not required. Thus, many variations on the above-described operational parameters are contemplated. For example, nanoseconds pulse length delivery may be used instead of microseconds.

Also as described, conductive or other gels may be used with the above-described systems and methods. Such gels, such as, for example, an ultrasound gel, can have active ingredients such as biotin and vitamins that can be delivered into the cells through PEF, in addition to the hair stimulation effect of the PEF directly. Also, such gel, which may include ultrasound gel, may contain no vitamins or biotin. In one configuration, the use of gel with the above-described systems and methods was tested using parameters of 270V, 300 pulses, 270 us pulse length duration and 500V, 200 pulses, 70 us pulse length duration. The tests confirmed that gel can be used to further facilitate treatment of skin through existing hair.

The present invention has been described in terms of one or more preferred embodiments, and it should be appreciated that many equivalents, alternatives, variations, and modifications, aside from those expressly stated, are possible and within the scope of the invention.

1. A system for stimulating hair growth, the system comprising:
a stimulating unit, the stimulating unit including:
at least one electrode,
a pulser in communication with the electrode and configured to provide an electronic pulse to the electrode;
a power source configured to provide power to the pulser; and
a control unit configured to communicate at least one stimulation parameter to the pulser, wherein the at least one stimulation parameter is selected to cause hair follicles proximate to the electrode to enter an anagen stage.

2. The system of claim 1, further comprising a user input and wherein the control unit is a computer configured to receive the stimulation parameters from the user input.

3. The system of claim 1, wherein the control unit is integral with the stimulating unit.

4. The system of claim 3, wherein the control unit is programmed with predetermined stimulation parameters.

5. The system of claim 1, further comprising an article in which the stimulating unit is integrated.

6. The system of claim 5, wherein the article is formed as a helmet, including a helmet configured to fit a scalp of a user, a hand-held device, including a wand or comb.

7. The system of claim 1, wherein the at least one stimulation parameter is one of voltage, pulse length, pulse frequency, and/or pulse number.

8. The system of claim 7, wherein the range of the voltage parameter is about 30 volts to 500 volts.

9. The system of claim 7, wherein the range of the pulse number parameter is about 200 to 900 pulses.

10. The system of claim 7, wherein the range of the pulse length parameter is about 10 microseconds to 270 microseconds.

11. A method for stimulating hair growth, the method comprising:
placing at least one electrode of a stimulating unit on a region of a user;
delivering at least one electric pulse to the at least one electrode according to at least one pulse parameter, wherein the at least one pulse parameter is selected to cause hair follicles proximate to the region of the user to enter an anagen stage;
terminating the delivery of the electric pulses when a predetermined number of pulses have been delivered, wherein the predetermined number is selected to cause hair follicles proximate to the region of the user to enter the anagen stage; and
removing the at least one electrode from the region of the user.

12. The method of claim 11, further including the step of determining the at least one pulse parameter.

13. The method of claim 12, further including the step of manually providing the at least one pulse parameter to a pulser.

14. The method of claim 12, wherein the at least one pulse parameter is one of voltage, pulse length, pulse frequency, and/or pulse number.

15. The method of claim 11, wherein the step of placing the at least one electrode is completed by placing an article comprising the at least one electrode on the region of the user.

16. The method of claim 11, further comprising arranging a conductive material about the at least one electrode and the region of the user.

17. The method of claim 16, wherein the conductive material includes a gel.

18. The method of claim 17, wherein the gel includes an ultrasound gel.

19. The method of claim 16, wherein the conductive material is conductive to at least one of electricity and acoustic signals.

20. The method of claim 16, wherein the material includes one of vitamins and biotins.
